(19) United States
(12) Patent Application Publication
(10) Pub. No.: US 2018/0059092 A1
(43) Pub. Date: Mar. 1, 2018

(54) HAND-HELD TEST METER WITH SMOOTH DIMPLED ELECTRICAL CONTACTS, CONTACT STOPPER RIBS, AND METAL SHIELD PLATE

(71) Applicant: Cilag GmbH International, Zug (CH)

(72) Inventor: Kevin CHONG, Inverness (GB)

(21) Appl. No.: 15/246,702

(22) Filed: Aug. 25, 2016

(51) Int. Cl. G01N 33/49; G01N 33/487; G01N 27/327

(52) U.S. Cl. CPC G01N 33/49; G01N 27/3273; G01N 27/3272; G01N 33/48707

(57) ABSTRACT

A hand-held test meter for use with an analytical test strip for the determination of an analyte in a bodily fluid sample includes a test meter housing with a housing outer surface and a distal end, a PCB disposed within the test meter housing; a SPC disposed in the test meter housing, and a metal shield plate securely attaching the strip port connector to the PCB. The SPC includes a SPC housing with at least one contact stopper rib, and plurality of electrical contacts (e.g., electrical contacts with a T-bar shaped distal end) disposed in the SPC housing. Each of the plurality of electrical contacts has a distal end and a test strip contact surface (TSCS). The TSCS of the electrical contact is configured for operable contact with an analytical test strip received in the SPC and the TSCS of the electrical contact is a smooth dimpled surface.

### BACKGROUND OF THE INVENTION

### Field of the Invention

The present invention relates, in general, to medical devices and, in particular, to test meters for use with analytical test strips and related methods.

### Description of Related Art

The determination (e.g., detection and/or concentration measurement) of an analyte in a fluid sample is of particular interest in the medical field. For example, it can be desirable to determine glucose, ketone bodies, cholesterol, lipoproteins, triglycerides, acetaminophen and/or HbA1c concentrations in a sample of a bodily fluid such as urine, blood, plasma or interstitial fluid. Such determinations can be achieved using analytical test strips along with an associated test meter.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, in which like numerals indicate like elements, of which:

FIGS. 1A and 1B are simplified perspective views of a hand-held test meter according to an embodiment of the present invention without an analytical test strip received therein (FIG. 1A) and with an analytical test strip received therein (FIG. 1B), respectively;

FIG. 2 is a simplified perspective view of a portion of the hand-held test meter of FIG. 1;

FIG. 3 is a simplified perspective rear-side (proximal-side) view of a strip port connector (SPC) as employed in an embodiment of the present invention;

FIG. 4 is a simplified perspective front-side (distal-side) view of the strip port connector of FIG. 3;

FIG. 5 is a simplified exploded view of a strip port connector (SPC) of FIG. 3;

FIG. 6 is a simplified perspective front-side (distal-side) view of the strip port connector of FIG. 3 attached to a printed circuit board (PCB) as employed in an embodiment of the present invention;

FIG. 7 is a simplified bottom view of the strip port connector of FIG. 3;

FIG. 8 is a simplified perspective view of an electrical contact as can be employed in embodiments of the present invention;

FIGS. 9A and 9B are simplified perspective views of a distal portion of the electrical contact of FIG. 8;

FIGS. 10A and 10B are simplified perspective cross-sectional views along the centerline of the strip port connector of FIG. 3 without an analytical test strip received therein (FIG. 10A) and with an analytical test strip TS received therein (FIG. 10B),

FIG. 11A and FIG. 11B are simplified cross-sectional bottom perspective views of a portion of the strip port connector of FIG. 3 in the absence of a received analytical test strip (FIG. 11A) and in the presence of a received analytical test strip (FIG. 11B), and

FIG. 12 is a flow diagram depicting stages in a method for employing a test meter for use with an analytical test strip in the determination of an analyte in a bodily fluid sample according to an embodiment of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein.

In general, hand-held test meters for use with an analytical test strip (such as an electrochemical-based analytical test strip) for the determination of an analyte (for example, glucose) in a bodily fluid sample (e.g., a whole blood sample) according to the present invention include a test meter housing with a housing outer surface and a distal end, a printed circuit board (PCB) disposed within the test meter housing, a strip port connector (SPC) disposed in the test meter housing, and a metal shield plate securely attaching the strip port connector to the printed circuit board. The strip port connector includes a strip port connector housing with at least one contact stopper rib, and plurality of electrical contacts disposed in the strip port connector housing.

Each of the plurality of electrical contacts has a distal end and a test strip contact surface. Moreover, the test strip contact surface of the electrical contact is configured for operable contact with an analytical test strip received in the strip port connector and the test strip contact surface of the electrical contact is a smooth dimpled surface. In addition, the distal end of the electrical contact and the contact stopper rib are configured such that, in the absence of a received analytical test strip, the stopper rib physically limits travel of the distal end of the electrical contact and thereby restricts the electrical contact to a predetermined position within the strip port connector.

Hand-held test meters according to the present invention are beneficial in that the smooth dimpled surface of the test strip contact surface increases the reliability of contact with an inserted test strip. In addition, the metal shield plate is beneficial in that it enables strip port connectors employed in embodiments of the present invention to be attached to printed circuit boards employed in the embodiments using standard, low cost, automated surface mount techniques (such as the same surface mount techniques employed to mount integrated circuits (ICs) and other electronic components onto the PCB).

The contact stopper rib employed in embodiments of the present invention beneficially ensures that the return position of the electrical contact(s) after deflection (i.e., after strip removal) is always at the same predetermined position. In one embodiment, the electrical contact has a distal end with a T-bar shape that beneficially determines the return position by mechanical interaction with the stopper rib.

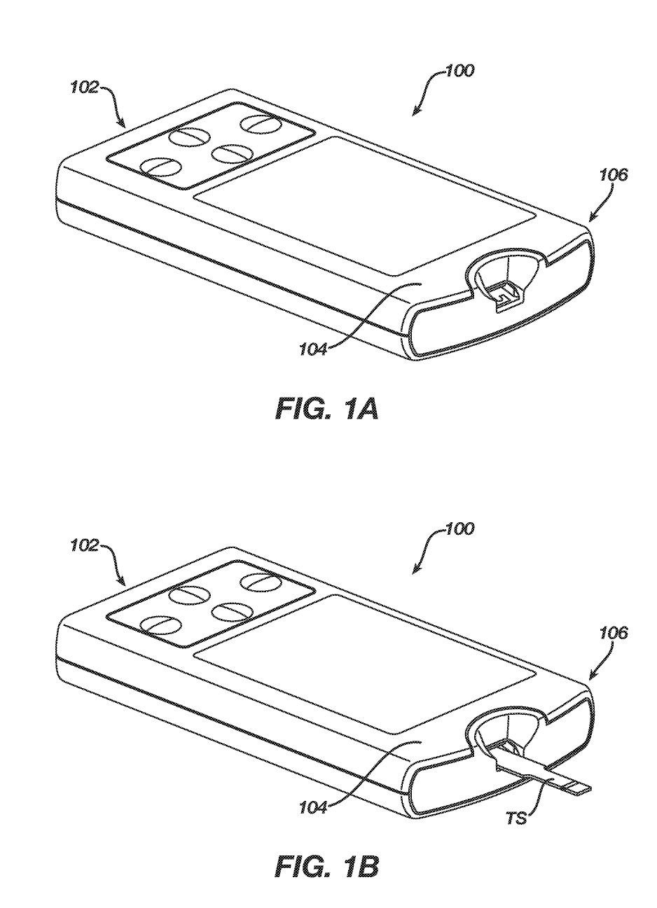

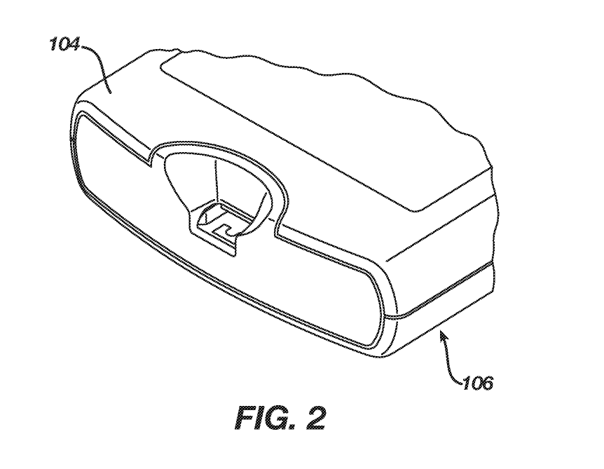

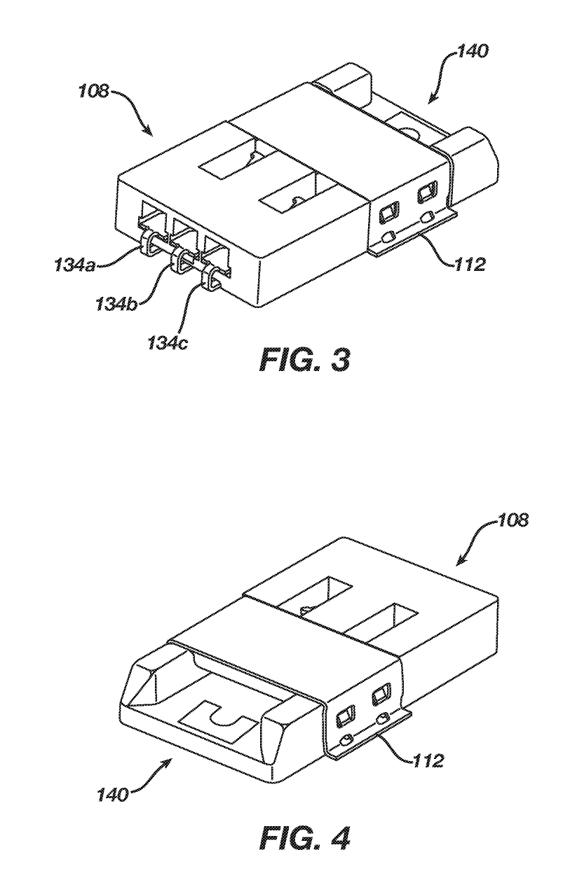

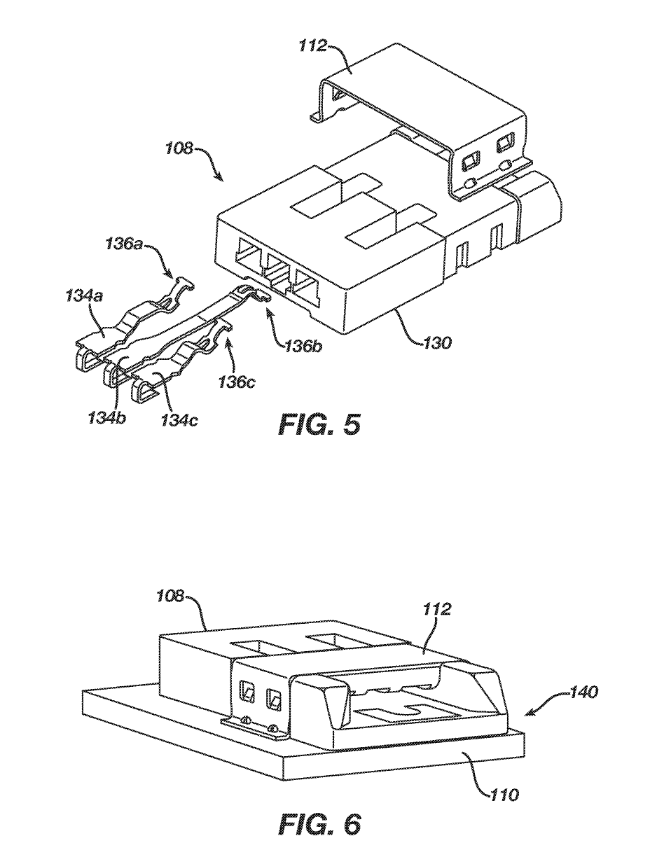

FIGS. 1A and 1B area simplified perspective views of a hand-held test meter 100 with and without an analytical test strip received therein according to an embodiment of the present invention. FIG. 2 is a simplified perspective angled view of a portion of hand-held test meter 100. FIG. 3 is a simplified perspective rear-side (proximal-side) view of a strip port connector (SPC) 108 as employed in an embodiment of the present invention. FIG. 4 is a simplified perspective front-side (distal-side) view of strip port connector 108. FIG. 5 is a simplified exploded view of strip port connector (SPC) 108. FIG. 6 is a simplified perspective front-side (distal-side) view strip port connector 108 attached to a printed circuit board (PCB) 110.

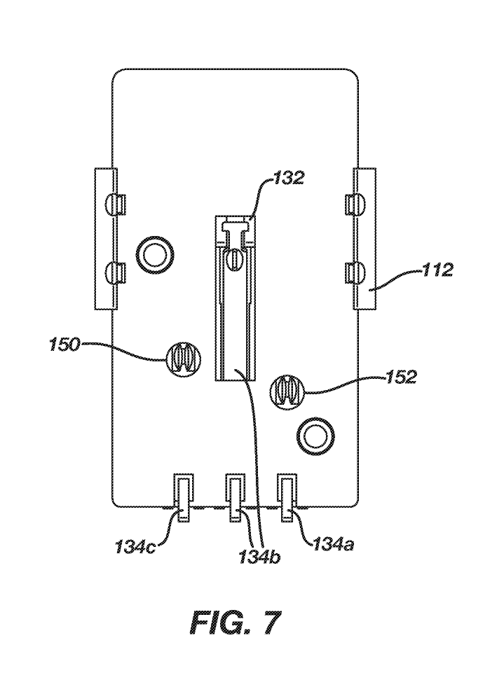

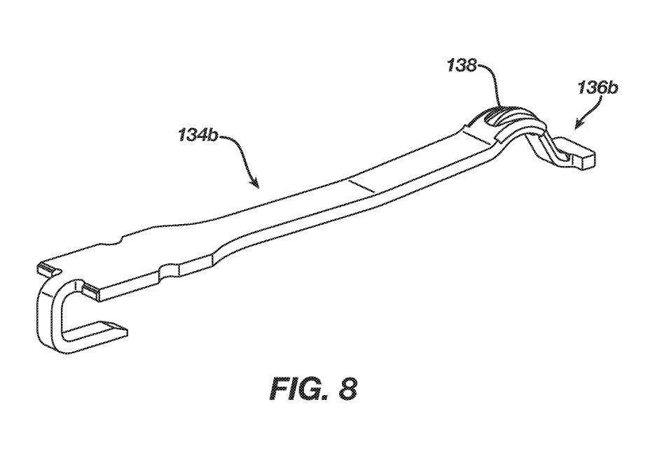

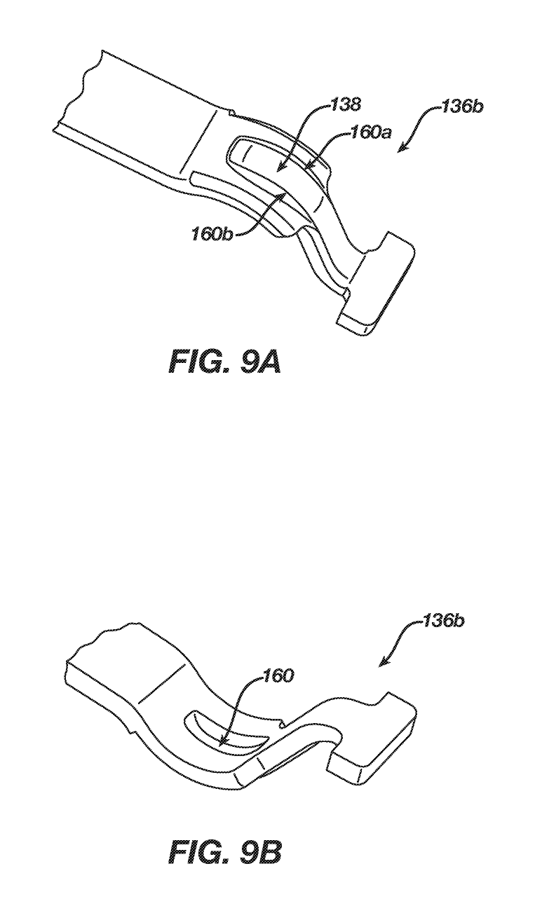

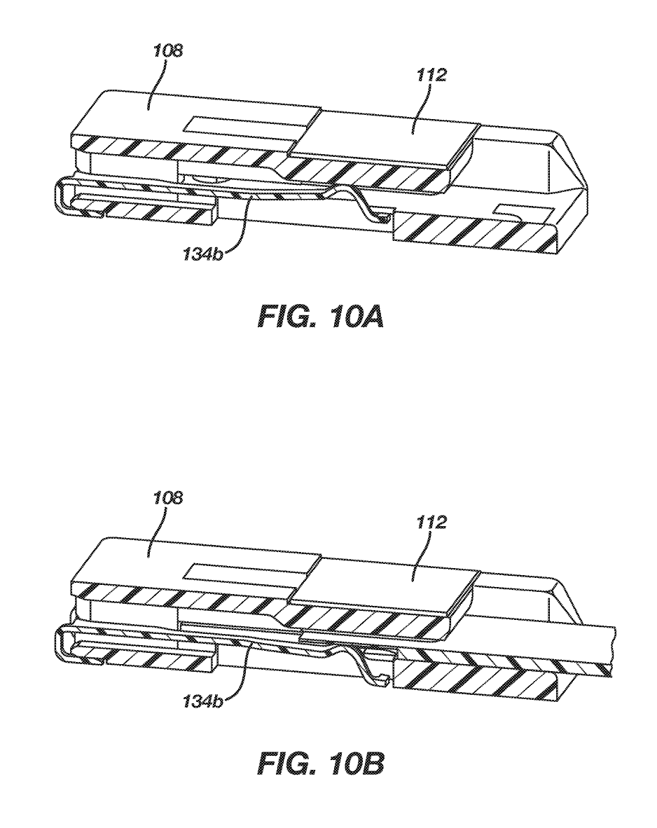

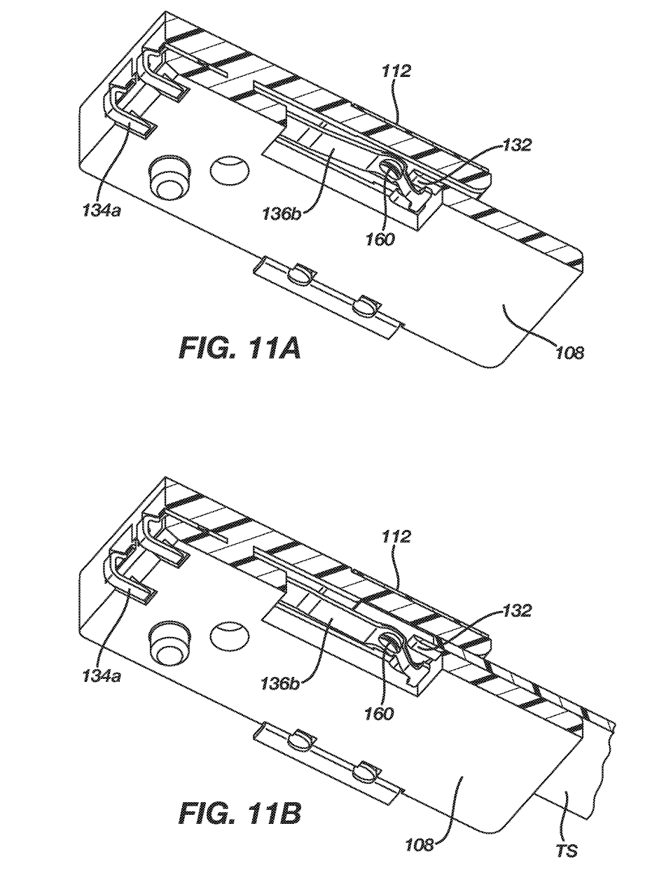

FIG. 7 is a simplified bottom view of strip port connector 108. FIG. 8 is a simplified perspective view of an electrical contact 134b as can be employed in embodiments of the present invention. FIGS. 9A and 9B are simplified perspective views of a distal portion of electrical contact 134b. FIGS. 10A and 10B are simplified cross-sectional views along the centerline of strip port connector 108 with FIG. 10B depicting an operably inserted (i.e., received) test strip. FIG. 11A and FIG. 11B are simplified cross-sectional bottom views of a portion of strip port connector 108 with and without an analytical test strip received therein.

Referring to FIGS. 1A through 11, hand-held test meter 100 for the determination of an analyte in a bodily fluid sample according to an embodiment of the present invention includes a test meter housing 102, with a housing outer surface 104 and a distal end 106, and a strip port connector 108 that is disposed within distal end 106 of test meter housing 102. Hand-held test meter 100 also includes a PCB 110 (see FIG. 6 in particular) disposed within test meter housing 102 and a metal shield plate 112.

Test meter housing 102 can be formed of any suitable material including, for example, polycarbonate materials, thermo-plastic polymeric materials, glass-reinforces thermoplastic polymeric materials, acrylonitrile butadiene styrene (ABS) materials, liquid-crystal polymer, polymeric materials in general, and combinations thereof. Test meter housing 102 can be formed, for example, using standard injection molding techniques.

Strip port connector (SPC) 108 is disposed in test meter housing 102 and includes a strip port connector housing 130 with at least one contact stopper rib 132 (see FIGS. 11A and 11B in particular) and a plurality of electrical contacts 134a, 134b and 134c disposed at least partially in strip port connector housing 130. Each of the plurality of electrical contacts having a distal end 136a-136c and a test strip contact surface such as test strip contact surface 138 of electrical contact 134b. The test strip contact surfaces of the electrical contact are configured for operable contact with an analytical test strip received in the strip port connector. In addition, the test strip contact surface of the electrical contact is a smooth dimpled surface (see FIGS. 8, 9A, and 9B in particular). Strip port connector 108 includes a distal portion 140.

Electrical contacts 134a, 134b and 134c are configured for operable electrical contact with an analytical test strip received within the strip port opening (see, for example, FIG. 10B and FIG. 11B). Electrical contacts employed in embodiments of the present invention can be formed of any suitable material including, for example Phosphor bronze plated first with nickel and then plated with gold to provide beneficial electrical conductivity.

Once apprised of the present disclosure, one skilled in the art will recognize that SPC 108 is an electromechanical component hand-held test meter 100 configured to operably interface with test strips. Conventional strip port connectors are described in, for example, US Patent Application Publication No. US 2012-0252133 entitled “Test Meter with a Strip Port Connector Configured for Fluid Entrapment” and U.S. patent application Ser. No. 14/911,498 entitled “Watertight Casing with Integrated Electrical Contacts”, each of which is hereby incorporated in full be reference.

Metal shield plate 112 is configured to securely attach strip port connector 108 to PCB 110 by, for example, standard surface mount techniques. Metal shield 112 is also configured to provide a flat upper surface for operable engagement during surface mount technology (SMT) pick-and-place operations and, optionally, configured to act as an Electrostatic Discharge (ESD) shield for the underlying PCB. The ESD shield capabilities are provided, for example, by connecting metal shield plate 112 to a ground circuit of PCB 110.

Metal shield plate 112 can be formed of any suitable material including, for example, Stainless Steel (e.g., SUS304 1/2H grade stainless steel with thickness of 0.15 mm+/−0.01 mm) plated with Nickel and Tin. The nickel and tin plating of such a metal shield plate provide for soldering of the metal shield plate to the PCB using conventional solder reflow techniques.

As depicted in FIG. 7, strip port connector housing 130 includes laser alignment opens 150 and 152 (see FIG. 7). Laser alignment openings 150 and 152 are configured for a laser beam to pass therethrough during automated alignment checking of, for example, electrical contacts 134a, 134b and 134c.

In hand-held test meter 100, the distal end of the electrical contact and the contact stopper rib are configured such that in the absence of a received analytical test strip the stopper rib physically limits travel of the distal end of the electrical contact and thereby restricts the electrical contact to a predetermined position within the strip port connector. FIGS. 7 and 11A depict the distal end of contact 134b stopped by contact stopper rib 132, while the dashed circle of FIG. 10A encompasses the predetermined position to which the distal end is limited (i.e., the return position) despite the electrical contact having a relatively strong spring force (achieved, for example, by a predetermined bend(s) in the electrical contact a predetermined distance from the distal end thereof). The relatively strong (and thus beneficial) spring force can be, for example, in the range of 1 Newton to 4 Newtons. Moreover, predetermined placement of the stopper rib(s) enables a predetermined setting (i.e., tuning) of the contact force against an inserted analytical test strip. In addition, the stopper rib(s) contributes to a beneficially small SPC since deflection of the electrical contact(s) upon insertion of an analytical test strip is restricted to a beneficially minimal distance.

In the embodiment of hand-held test meter 100, the distal end of the electrical contacts have a T-bar shape (see FIGS. 9A, 10A, 10B, 11A and 11B in particular) that securely restricts the electrical contact position such that the return position after deflection (i.e., after strip removal) is always back to a predetermined position. This deflection auto-recovery (auto-return) function is achieved despite a relatively high pre-bend (pre-load) angle on the metal contact design. In other words, the T-bar shape in conjunction with the stopper rib controls the return position of each metal contact.

In the embodiment of FIGS. 9A and 9B, the smooth dimpled contact surface is essentially defined by two burr-free smooth edges 160a and 160b. In this regards, the term “dimple” refers to a depression or indentation on a surface or, in other words, a concavity in the underside surface (see FIG. 9B) produced, for example, by pressing. The term “smooth” refers to a continuous surface contour that blends in seamlessly across the electrical contact, providing a smooth transition from the initiation of analytical test strip insertion till electrical connection is established at the dimpled contact surface (see FIG. 9B in particular). The radii of curvature of the dimple (i.e., the dimpled contact surface) can be any suitable radius. In the embodiment of FIGS. 9A and 9B, the smooth dimple defined by edges 160A and 160B is an elongated dimple running along the longitudinal axis of the electrical contact. Since the longitudinal axis is also the direction of analytical test strip insertion, the smooth dimpled surface provides for a beneficially smooth insertion of an analytical test strip.

Methods according to embodiments of the present invention are beneficial in that they provide a secure operable contact between an analytical test strip received in a strip port connector of a hand-held test meter and electrical contact(s) of the strip port connector. The benefits of a secure operable contact is achieved by employing electrical contacts with a smooth dimpled surface (also referred to herein as an electrical contact surface with a smooth dimpled surface) and/or an electrical contact that is operably positioned by a stopper rib of the hand-held test meter prior to insertion of the analytical test strip.

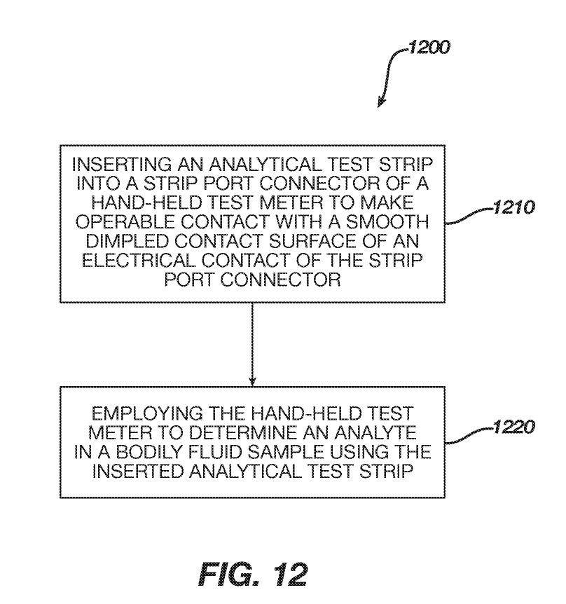

FIG. 12 is a flow diagram depicting stages in a method 1200 for employing a test meter for use with an analytical test strip in the determination of an analyte (such as glucose) in a bodily fluid sample (for example, a whole blood sample). Method 1200 includes, at step 1210, inserting an analytical test strip into a strip port connector of a hand-held test meter such that the inserted analytical test strip is received in the strip port connector by making operable contact with a plurality of electrical contacts of the strip port connector. In method 1200, each of the plurality of electrical contacts has a test strip contact surface. Moreover, the test strip contact surface of the electrical contact is configured for operable contact with an analytical test strip received in the strip port connector and is a smooth dimpled contact surface.

Method 1200 also includes employing the hand-held test meter to determine an analyte in a bodily fluid sample using an analytical test strip received in the strip port connector (see step 1220 of FIG. 12). The received analytical test strip can be, for example, an electrochemical-based analytical test strip.

Once apprised of the present disclosure, one skilled in the art will recognize that methods according to embodiments of the present invention including method 1200 can be readily modified to incorporate any of the techniques, benefits and characteristics of hand-held test meters according to embodiments of the present invention and described herein including, for example, the inclusion of at least one stopper rib in the hand-held test meter.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and methods within the scope of these claims and their equivalents be covered thereby.

What is claimed is:

1. A hand-held test meter for use with an analytical test strip for the determination of an analyte in a bodily fluid sample, the test meter comprising:
a test meter housing with a housing outer surface and a distal end;
a printed circuit board (PCB) disposed within the test meter housing;
a strip port connector (SPC) disposed in the test meter housing, the strip port connector including:
a strip port connector housing with at least one contact stopper rib;
a plurality of electrical contacts disposed in the strip port connector housing, each of the plurality of electrical contacts having a distal end and a test strip contact surface, wherein
the test strip contact surface of the electrical contact is configured for operable contact with an analytical test strip received in the strip port connector; and.
the test strip contact surface of the electrical contact is a smooth dimpled surface; and
a metal shield plate securely attaching the strip port connector to the printed circuit board;
and wherein the distal end of the electrical contact and the contact stopper rib are configured such that in the absence of a received analytical test strip the stopper rib physically limits travel of the distal end of the electrical contact and thereby restricts the electrical contact to a predetermined position within the strip port connector.

2. The hand-held test meter of claim 1 wherein the strip port connector (SPC) is attached to the printed circuit board (PCB) via solder connection between the PCB and the metal shield plate.

3. The hand-held test meter of claim 1 wherein the distal end of each of the plurality of electrical contacts is T-shaped.

4. The hand-held test meter of claim 1 wherein the smooth dimpled surface includes a dimple with smooth edges on either side of a longitudinal center line of the electrical contact.

5. The hand-held test meter of claim 1 wherein there are three electrical contacts.

6. The hand-held test meter of claim 1 wherein the SPC housing is formed of a polymeric material.

7. The hand-held test meter of claim 6 wherein the metal shield plate is secured to the SPC housing via snap fit between the SPC housing and the metal shield plate.

8. The hand-held test meter of claim 1 wherein SPC housing includes at least one laser alignment opening.

9. The hand-held test meter of claim 1 wherein the electrical contact has a predetermined elastic force due at least in part to a bend in the electrical contacts.

10. The hand-held test meter of claim 1 wherein the hand-held test meter and analytical test strip are configured for the determination of glucose in a whole blood sample.

11. The hand-held test meter of claim 1 wherein the analytical test strip is an electrochemical-based analytical test strip.

12. A method for employing a hand-held test meter for use with an analytical test strip in the determination of an analyte in a bodily fluid sample, the method comprising:
inserting an analytical test strip into a strip port connector of a hand-held test meter such that the inserted analytical test strip is received in the strip port connector by making operable contact with a plurality of electrical contacts of the strip port connector, each of the plurality of electrical contacts having a distal end and a test strip contact surface, wherein
the test strip contact surface of the electrical contact is configured for operable contact with an analytical test strip received in the strip port connector; and
the test strip contact surface of the electrical contact is a smooth dimpled surface; and
employing the test meter to determine an analyte in a bodily fluid sample using the analytical test strip inserted in the strip port connector.

13. The method of claim 12 wherein the hand-held test meter includes metal shield plate securely attaching the strip port connector to a printed circuit board of the hand-held test meter.

14. The method of claim 13 wherein the strip port connector (SPC) is attached to the printed circuit board (PCB) via solder connection between the PCB and the metal shield plate.

15. The method of claim 12 wherein the wherein the distal end of the electrical contact and a contact stopper rib of the strip port connector are configured such that in the absence of a received analytical test strip the stopper rib physically limits travel of the distal end of the electrical contact and thereby restricts the electrical contact to a predetermined position within the strip port connector.

16. The method of claim 12 wherein the distal end of each of the plurality of electrical contacts is T-shaped.

17. The method of claim 12 wherein the smooth dimpled surface includes a smooth dimple defined by smooth edges on either side of a longitudinal center line of the electrical contact.

18. The method of claim 12 wherein there are three electrical contacts.

19. The method of claim 12 wherein the SPC housing is formed of a polymeric material.

20. The method of claim 19 wherein the metal shield plate is secured to the SPC housing via snap fit between the SPC housing and the metal shield plate.

21. The method of claim 12 wherein strip port controller housing includes at least one laser alignment opening.

22. The method of claim 12 wherein the hand-held test meter and analytical test strip are configured for the determination of glucose in a whole blood sample.

23. The method of claim 12 wherein the analytical test strip is an electrochemical-based analytical test strip.
